(19) United States
(12) Patent Application Publication
(10) Pub. No.: US 2025/0026720 A1
(43) Pub. Date: Jan. 23, 2025

(54) METHOD AND DEVICE FOR MANUFACTURING TRIACETONAMINE

(71) Applicant: INDUSTRIAL TECHNOLOGY RESEARCH INSTITUTE, Hsinchu (TW)

(72) Inventors: Jyun-Wei HONG, Fangyuan Township (TW); Chien-Wei HSU, Houlong Township (TW)

(73) Assignee: INDUSTRIAL TECHNOLOGY RESEARCH INSTITUTE, Hsinchu (TW)

(21) Appl. No.: 18/758,970

(22) Filed: Jun. 28, 2024

Related U.S. Application Data
(60) Provisional application No. 63/513,608, filed on Jul. 14, 2023.

(30) Foreign Application Priority Data
Mar. 5, 2024 (TW) 113107911

(51) Int. Cl. C07D 211/74; B01D 3/10; B01J 31/02

(52) U.S. Cl. CPC C07D 211/74; B01D 3/10; B01J 31/0238; B01J 31/0239

(57) ABSTRACT

A method of manufacturing triacetonamine includes (a) introducing an acetone and an ammonia into a first reactor in the presence of a first acidic catalyst to form an acetonin; (b) introducing the acetonin and water into a second reactor in the presence of a second acidic catalyst to form a triacetonamine and side products, wherein the side products include diacetone alcohol, diacetone amine, mesityl oxide, 2,2,4,6-tetramethyl-2,3-dihydropyridine, or a combination thereof; (c) separating the triacetonamine and the side products by distillation under reduced pressure; (d) introducing the side products and water into a third reactor to heat and crack the side products in the presence of an amphiphilic catalyst to form acetone; and (e) introducing the acetone obtained from step (d) into the first reactor.

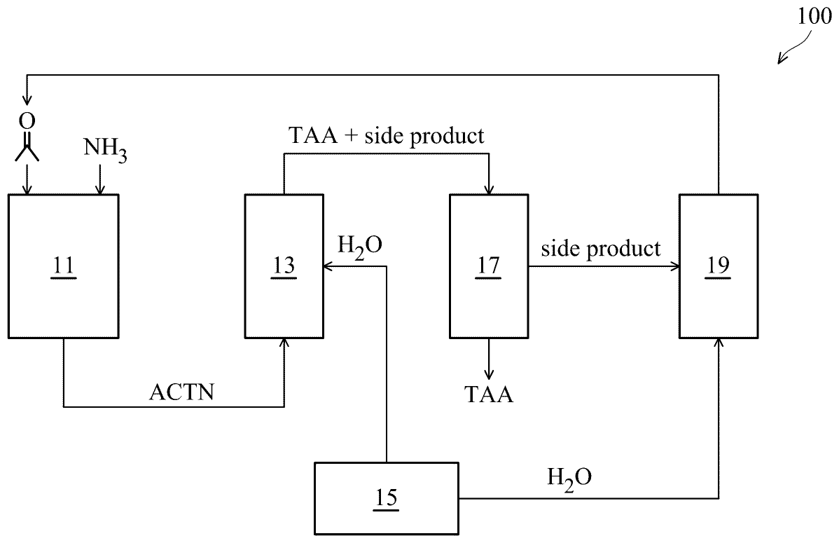

### CROSS REFERENCE TO RELATED APPLICATIONS

This application claims the benefit of U.S. Provisional Application No. 63/513,608, filed on Jul. 14, 2023, the entirety of which is incorporated by reference herein. The present application is based on, and claims priority from, Taiwan Application Serial Number 113107911, filed on Mar. 5, 2024, the disclosure of which is hereby incorporated by reference herein in its entirety.

### TECHNICAL FIELD

The technical field relates to a method of manufacturing triacetonamine, and in particular it relates to cracking side products to recycle acetone.

### BACKGROUND

Antioxidants or (and) light stabilizers are often added during plastic processing and molding to slow down side reactions such as photo degradation, thermal degradation, or deliquescence, so as to extend the lifespan and improve the weather resistance of the finished product. The triacetonamine molecule is a crucial precursor for light stabilizers. A conventional method of synthesizing triacetonamine is homogeneously reacting acetone, ammonia, and ammonium nitrate in a single step. Despite the acceptable yield of the triacetonamine product, the numerous side products produced during the triacetonamine synthesis, cause problems in waste treatment and in the subsequent purification steps.

Accordingly, a novel method of synthesizing triacetonamine is called for to address the issue of the side products.

### SUMMARY

One embodiment of the disclosure provides a method of manufacturing triacetonamine, including: (a) introducing an acetone and an ammonia into a first reactor in the presence of a first acidic catalyst to form an acetonin; (b) introducing the acetonin and water into a second reactor in the presence of a second acidic catalyst to form a triacetonamine and side products, wherein the side products are diacetone alcohol, diacetone amine, mesityl oxide, 2,2,4,6-tetramethyl-2,3-dihydropyridine, or a combination thereof; (c) separating the triacetonamine and the side products by distillation under reduced pressure; (d) introducing the side products and water into a third reactor to heat and crack the side products in the presence of an amphiphilic catalyst to form acetone; and (e) introducing the acetone obtained from step (d) into the first reactor.

One embodiment of the disclosure provides a device for manufacturing triacetonamine. The device for manufacturing triacetonamine includes a first reactor, a second reactor, a distiller, and a third reactor. The first reactor contains a first acidic catalyst. The first reactor receives acetone and ammonia to form an acetonin. The second reactor contains a second acidic catalyst. The second reactor is connected to the first reactor and a water source. The second reactor receives the acetonin and water, respectively, to form a triacetonamine and side products. The side products are diacetone alcohol, diacetone amine, mesityl oxide, 2,2,4,6-tetramethyl-2,3-dihydropyridine, or a combination thereof. The distiller is connected to the second reactor for receiving the triacetonamine and the side products and for separating the triacetonamine and the side products by distillation under reduced pressure. The third reactor contains an amphiphilic catalyst. The third reactor is connected to the distiller and the water source for receiving the side products and water, respectively, to heat and crack the side products to form an acetone. The third reactor is connected to the first reactor for introducing the acetone in the third reactor into the first reactor.

A detailed description is given in the following embodiments with reference to the accompanying drawings.

### BRIEF DESCRIPTION OF THE DRAWINGS

The disclosure can be more fully understood by reading the subsequent detailed description and examples with references made to the accompanying drawings, wherein:

FIGURE shows a device for manufacturing triacetonamine in an embodiment of the disclosure.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

One embodiment of the disclosure provides a device 100 of manufacturing triacetonamine, as shown in Figure. The device 100 includes a first reactor 11 containing a first acidic catalyst and receiving an acetone and an ammonia to form an acetonin (ACTN, having a chemical structure of

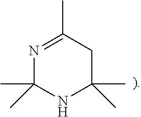

In some embodiments, the first acidic catalyst can be Amberlyst 15 (commercially available from ACROS, Alfa Aesar, or DuPont).

The device 100 also includes a second reactor 13 containing a second acidic catalyst and connected to the first reactor 11 and a water source 15 for receiving the acetonin and water, respectively, to form a triacetonamine (TAA, having a chemical structure of

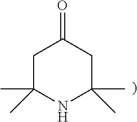

and side products. The second acidic catalyst can be Amberlyst 15 (commercially available from ACROS, Alfa Aesar, or DuPont). In some embodiments, the first acidic catalyst can be as same as the second acidic catalyst. In some embodiments, the first acidic catalyst can be different from the second acidic catalyst. The side products are diacetone alcohol (DAA, having a chemical structure of

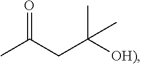

diacetone amine (DAAM, having a chemical structure of

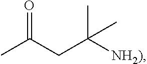

mesityl oxide (MO, having a chemical structure of

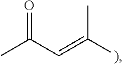

2,2,4,6-tetramethyl-2,3-dihydropyridine (TMDP, having a chemical structure of

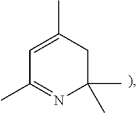

or a combination thereof.

The device 100 also includes a distiller 17 connected to the second reactor for receiving TAA and the side products and separating TAA and the side products by distillation under reduced pressure.

The device 100 also includes a third reactor 19 containing an amphiphilic catalyst and connected to the distiller 17 and the water source 15 for receiving the side products and water, respectively. The third reactor 19 may heat and crack the side products to form an acetone. The third reactor 19 is further connected to the first reactor 11 for introducing the acetone in the third reactor into the first reactor 11. In some embodiments, the amphiphilic catalyst has a chemical structure of

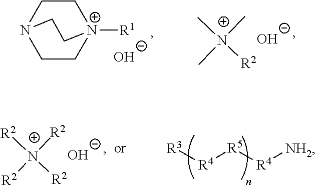

wherein R¹ is C₄-10 alkyl group, each of R² is independently C₄₋₁₆ alkyl group, R³ is —NH₂ or —OH, R⁴ is C₂₋₆ alkylene group, R⁵ is —NH— or —O—, and n=0 to 4. If R¹ is a lone pair electron (i.e. 1,4-Diazabicyclo [2.2.2]octane (DABCO)) or an alkyl group of low carbon numbers (e.g. ≤C₃), the side products cannot be efficiently cracked to form acetone. If the carbon number of R² is less than 4 (e.g. 1 to 3), the side products cannot be cracked to form acetone. If n is too large (e.g. >4), the amphiphilic catalyst will have a poor effect on cracking the side products to form the acetone. As shown in the experiments, the amphiphilic catalyst after repeated use still has a certain efficiency on cracking the side products without significant degradation.

One embodiment of the disclosure provides a method of manufacturing triacetonamine, including: (a) introducing an acetone and an ammonia into the first reactor 11 in the presence of the first acidic catalyst to form the acetonin. In some embodiments, the reaction temperature of the first reactor 11 is between 20° C. to 40° C. If the reaction temperature of the first reactor 11 is too low, the conversion rate of the acetone will be poor which may reduce the yield of acetonin. If the reaction temperature of the first reactor 11 is too high, the selectivity of acetonin will be decreased, and side products other than acetonin may be formed. In some embodiments, the acetone and the ammonia have a molar ratio of 1:1 to 20:1. If the amount of acetone is too high, the solid content of the acetonin will be lowered which may negatively influence the production capacity. If the amount of acetone amount is too low, the remaining unreacted ammonia will flow into the second reactor which may reduce the selectivity of triacetonamine.

Subsequently, (b) introducing the acetonin and water into the second reactor 13 in the presence of a second acidic catalyst to form triacetonamine and the side products, and the side products are diacetone alcohol, diacetone amine, mesityl oxide, 2,2,4,6-tetramethyl-2,3-dihydropyridine, or a combination thereof. In some embodiments, water can be provided from the water source 15 of the device 100. In some embodiments, the reaction temperature of the second reactor is between 60° C. to 90° C. If the reaction temperature of the second reactor 13 is too low, the conversion rate of the acetonin will be poor which may reduce the yield of triacetonamine. If the reaction temperature of the second reactor 13 is too high, the selectivity of triacetonamine will be decreased and form the side products other than triacetonamine.

Subsequently, (c) separating triacetonamine and the side products by distillation under reduced pressure. For example, the distillation under the reduced pressure can be performed in the distiller 17 of the device 100.

Subsequently, (d) introducing the side products and water into the third reactor 19 to heat and crack the side products in the presence of the amphiphilic catalyst to form acetone. For example, water can be provided from the water source 15 of the device 100. In some embodiments, the amphiphilic catalyst and the side products have a molar ratio of 1:5 to 1:40. If the amount of the amphiphilic catalyst is too low, the side products cannot be efficiently cracked to form the acetone. If the amount of amphiphilic catalyst is too high, the economic value of the process will be reduced. In some embodiments, the reaction temperature of the third reactor 19 ranges between 70° C. to 90° C. If the reaction temperature of the third reactor 19 is too low, the cracking efficiency will be reduced. If the reaction temperature of the third reactor is too high, the process will consume too much energy and the economic value of the process will be reduced.

Subsequently, (e) feeding back the acetone obtained from step (d) into the first reactor 11, so that the recycled acetone can be re-used to form the acetonin again, and the obtained acetonin and water can be re-used to form the triacetonamine and the side products again.

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLES

### Synthesis Example 1

100 g of 1,4-Diazabicyclo [2.2.2]octane (DABCO) and 14.7 g of 1-bromohexane were dissolved in 200 mL of ethyl acetate, and then stirred at room temperature to react for 72 hours. The ethyl acetate was removed after the reaction, resulting in a white solid residue. The white solid residue was dissolved in ethanol (200 mL), and 4.9 g of potassium hydroxide was added to the ethanol solution to precipitate potassium bromide. The potassium bromide was filtered out to collect a filtrate, and ethanol was removed from the filtrate to obtain a milky liquid as a product (18.65 g, 98%). The hydrogen spectrum of the product is shown below: ¹H NMR (400 MHz, D₂O, 298K): 3.35 (t, 6H), 3.15 (m, 8H), 1.71 (m, 2H), 1.29 (m, 6H), 0.83 (t, 3H). The chemical structure of the product is shown below:

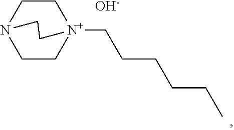

### Synthesis Example 2

19.5 g of hexadecyl trimethyl ammonium bromide and 3.0 g of potassium hydroxide were added into 200 mL of ethanol to precipitate potassium bromide. After stirring for 1 hour, the potassium bromide was filtered out to collect a filtrate, and the solvent was removed from the filtrate to obtain a pale yellow liquid as a product (15.91 g, 99%). The hydrogen spectrum of the product is shown below: ¹H NMR (400 MHz, D₂O, 298K): 3.32 (t, 2H), 3.12 (s, 9H), 1.75 (m, 2H), 1.31 (m, 26H), 1.87 (t, 3H). The chemical structure of the product is shown below:

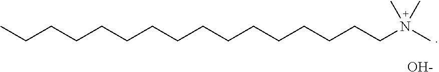

### Synthesis Example 3

5.0 g of DABCO and 4.9 g of 1-bromoethane were dissolved in 100 mL of ethyl acetate, and then stirred at room temperature to react for 72 hours. The ethyl acetate was removed after the reaction, resulting in a white solid residue. The white solid residue was dissolved in ethanol (100 mL), and 2.4 g of potassium hydroxide was added to the ethanol solution to precipitate potassium bromide. The potassium bromide was filtered out to collect a filtrate, and ethanol was removed from the filtrate to obtain a milky liquid as a product. The hydrogen spectrum of the product is shown below: ¹H NMR (400 MHz, D₂O, 298K) 3.39 (m, 8H), 3.24 (t, 6H), 1.37 (t, 3H). The chemical structure of the product is shown below:

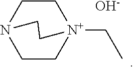

### Synthesis Example 4

5.0 g of DABCO and 5.5 g of 1-bromopropane were dissolved in 100 mL of ethyl acetate, and then stirred at room temperature to react for 72 hours. The ethyl acetate was removed after the reaction, resulting in a white solid residue. The white solid residue was dissolved in ethanol (100 mL), and 2.4 g of potassium hydroxide was added to the ethanol solution to precipitate potassium bromide. The potassium bromide was filtered out to collect a filtrate, and ethanol was removed from the filtrate to obtain a milky liquid as a product. The hydrogen spectrum of the product is shown below: ¹H NMR (400 MHz, D₂O, 298K): 3.42 (t, 6H), 3.23 (m, 8H), 1.80 (q, 2H), 0.99 (t, 3H). The chemical structure of the product is shown below:

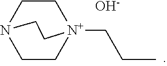

### Synthesis Example 5

5.0 g of DABCO and 8.6 g of 1-bromooctane were dissolved in 100 mL of ethyl acetate, and then stirred at room temperature to react for 72 hours. The ethyl acetate was removed after the reaction, resulting in a white solid residue. The white solid residue was dissolved in ethanol (100 mL), and 2.4 g of potassium hydroxide was added to the ethanol solution to precipitate potassium bromide. The potassium bromide was filtered out to collect a filtrate, and ethanol was removed from the filtrate to obtain a milky liquid as a product. The hydrogen spectrum of the product is shown below: ¹H NMR (400 MHz, D₂O, 298K): 3.43 (t, 6H), 3.25 (m, 8H), 1.35 (m, 10H), 0.91 (t, 3H). The chemical structure of the product is shown below:

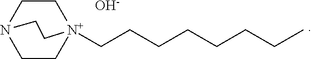

### Example 1

100 g of mesityl oxide (MO), 25 g of diacetone alcohol (DAA), 100 g of water, and 15.7 g of the product in Synthesis Example 1 were mixed in a round bottle with a dean-stark device and then heated to 85° C. to react for 3 hours. The reaction product was distilled to obtain 125.6 g of acetone (yield=87%).

Subsequently, 100 g of MO, 25 g of DAA, 100 g of water, and the product of Synthesis Example 1 that was used once were mixed in a round bottle with a dean-stark device and then heated to 85° C. to react for 3 hours. The reaction product was distilled to obtain 127.5 g of acetone (yield=89%).

Subsequently, 100 g of MO, 25 g of DAA, 100 g of water, and the product of Synthesis Example 1 that was used twice were mixed in a round bottle with a dean-stark device and then heated to 85° C. to react for 3 hours. The reaction product was distilled to obtain 123.2 g of acetone (yield=86%).

Accordingly, the product in Synthesis Example 1 after repeated use still had a certain efficiency on cracking MO and DAA without significant degradation.

### Example 2

100 g of MO, 25 g of DAA, 100 g of water, and 16.5 g of the product in Synthesis Example 2 were mixed in a round bottle with a dean-stark device and then heated to 85° C. to react for 3 hours. The reaction product was distilled to obtain 124.1 g of acetone (yield=85%).

### Example 3

100 g of MO, 25 g of DAA, 100 g of water, and 12.0 g of the product in Synthesis Example 5 were mixed in a round bottle with a dean-stark device and then heated to 85° C. to react for 3 hours. The reaction product was distilled to obtain 132.1 g of acetone (yield=92%).

### Example 4

100 g of MO, 25 g of DAA, 100 g of water, and 25.0 g of an aqueous solution of tetrabutylammonium hydroxide (TBAH, 40 wt %) were mixed in a round bottle with a dean-stark device and then heated to 85° C. to react for 3 hours. The reaction product was distilled to obtain 120.6 g of acetone (yield=84%).

### Example 5

100 g of MO, 25 g of DAA, 100 g of water, and 10.0 g of monoethanolamine (MEA) were mixed in a round bottle with a dean-stark device and then heated to a temperature range of 80° C. to 85° C. to react for 2 hours. The reaction product was distilled to obtain 123.4 g of acetone (yield=85%).

### Example 6

100 g of MO, 25 g of DAA, 100 g of water, and 10 g of triethylenetetramine (TETA) were mixed in a round bottle with a dean-stark device and then heated to a temperature range of 80° C. to 85° C. to react for 2 hours. The reaction product was distilled to obtain 124.3 g of acetone (yield=85%).

### Comparative Example 1

100 g of MO, 25 g of DAA, 100 g of water, and 10 g of Jeffamine D-230 (commercially available from Huntsman, having a chemical structure of

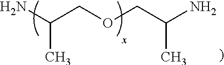

were mixed in a round bottle with a dean-stark device and then heated to a temperature range of 85° C. to 90° C. to react for 6 hours. The reaction product was distilled to obtain 21.4 g of acetone (yield=15%).

### Comparative Example 2

100 g of MO, 25 g of DAA, 100 g of water, and 9 g of ammonia solution (30 wt %) were mixed in a round bottle with a dean-stark device and then heated to 85° C. to react for 4 hours. The reaction product was distilled with no acetone obtained.

### Comparative Example 3

100 g of MO, 25 g of DAA, 100 g of water, and 10 g of NaOH were mixed in a round bottle with a dean-stark device and then heated to 85° C. to react for 3 hours. The reaction product was distilled to obtain 102.1 g of acetone (yield=71%).

### Comparative Example 4

100 g of MO, 25 g of DAA, 100 g of water, and 10 g of DABCO were mixed in a round bottle with a dean-stark device and then heated to 85° C. to react for 3 hours. The reaction product was distilled with no acetone obtained.

### Comparative Example 5

100 g of MO, 25 g of DAA, 100 g of water, and 10 g of decanediamine were mixed in a round bottle with a dean-stark device and then heated to 85° C. to react for 3 hours. The reaction product was distilled to obtain 23.2 g of acetone (yield=16%).

### Comparative Example 6

100 g of MO, 25 g of DAA, 100 g of water, and 10.0 g of the product in Synthesis Example 3 were mixed in a round bottle with a dean-stark device and then heated to 85° C. to react for 3 hours. The reaction product was distilled to obtain 41.0 g of acetone (yield=29%).

### Comparative Example 7

100 g of MO, 25 g of DAA, 100 g of water, and 10.0 g of the product in Synthesis Example 4 were mixed in a round bottle with a dean-stark device and then heated to 85° C. to react for 3 hours. The reaction product was distilled to obtain 51.3 g of acetone (yield=36%).

### Example a-1

A fixed bed reactor was set to have an inner diameter of 12.7 mm and a tube length of 760 mm. 20 mL of sulfonic acid resin Amberlyst 15 (commercially available from ACROS, Alfa Aesar, or DuPont) was filled into the reactor to serve as an acidic catalyst. The fixed bed reactor was maintained at a temperature of 30° C., while the pressure was maintained at 10 kg/cm².

Acetone and liquid ammonia (molar ratio=1.4:1) were introduced into the fixed bed reactor, the liquid hourly space velocity (LHSV) of the acetone was 1.15 h⁻¹, and the LHSV of the liquid ammonia was 0.25 h⁻¹. The crude product flowing out of the fixed bed reactor was separated into gas and liquid to obtain crude acetonin (liquid). The crude acetonin was analyzed by gas chromatography. The analysis results showed that the selectivity of the acetonin was 100% and the yield of the acetonin was 51%.

70 g of the acetonin crude, 3.15 g of water (4.5 wt %), and 8 g of Amberlyst 15 were added into an autoclave reactor, then heated to 75° C. and kept at a pressure of 10 kg/cm² to react for 6 hours to obtain a crude triacetonamine (TAA). The crude TAA was analyzed by gas chromatography, which showed the yield of TAA was 4% and the rest were side products.

### Example a-2

Acetone and liquid ammonia (molar ratio=2.8:1) were introduced into the fixed bed reactor in Example a-1, the LHSV of the acetone was 1.15 h⁻¹, and the LHSV of the liquid ammonia was 0.13 h⁻¹. The crude product flowing out of the fixed bed reactor was separated into gas and liquid to obtain the crude acetonin (liquid). The crude acetonin was analyzed by gas chromatography. The analysis results showed that the selectivity of acetonin was 100% and the yield of acetonin was 77%.

70 g of the acetonin crude, 3.15 g of water (4.5 wt %), and 8 g of Amberlyst 15 were added into an autoclave reactor, then heated to 75° C. and kept at a pressure of 10 kg/cm² to react for 6 hours, to obtain a crude TAA. The crude TAA was analyzed by gas chromatography, which showed the yield of TAA was 45% and the rest were side products.

### Example a-3

Acetone and liquid ammonia (molar ratio=5.7:1) were introduced into the fixed bed reactor in Example a-1, the LHSV of the acetone was 1.15 h⁻¹, and the LHSV of the liquid ammonia was 0.06 h⁻¹. The crude product flowing out of the fixed bed reactor was separated into gas and liquid, to obtain the crude acetonin (liquid). The crude acetonin was analyzed by gas chromatography. The analysis results showed that the selectivity of the acetonin was 100% and the yield of the acetonin was 91%.

70 g of the acetonin crude, 3.15 g of water (4.5 wt %), and 8 g of Amberlyst 15 were added into an autoclave reactor, then heated to 75° C. and kept at a pressure of 10 kg/cm² to react for 6 hours, to obtain a crude TAA. The crude TAA was analyzed by gas chromatography, which showed the yield of TAA was 84% and the rest were side products.

### Example a-4

Acetone and liquid ammonia (molar ratio=8.1:1) were introduced into the fixed bed reactor in Example a-1, the LHSV of the acetone was 1.15 h⁻¹, and the LHSV of the liquid ammonia was 0.04 h⁻¹. The product flowing out of the fixed bed reactor was separated into gas and liquid to obtain the crude acetonin (liquid). The crude acetonin was analyzed by gas chromatography. The analysis results showed the selectivity of acetonin was 100% and the yield of acetonin was 98%.

70 g of the acetonin crude, 3.15 g of water (4.5 wt %), and 8 g of Amberlyst 15 were added into an autoclave reactor, then heated to 75° C. and kept at a pressure of 10 kg/cm² to react for 6 hours to obtain a crude TAA. The TAA crude was analyzed by gas chromatography, which showed that the yield of TAA was 93% and the rest were side products.

### Example a-5

The crude TAA in Example a-3 was distilled to obtain a light-boiling substance. 25 g of the light-boiling substance, 20 g of water, and 4 g of the product of Synthesis Example 1 were mixed and then heated to a temperature range of 80° C. to 90° C. to react for 3 hours. The reaction product contained a large amount of acetone.

The recycled acetone and liquid ammonia (molar ratio=5.7:1) were introduced into the fixed bed reactor in Example a-1, the LHSV of the acetone was 1.15 h⁻¹, and the LHSV of the liquid ammonia was 0.06 h⁻¹. The product flowing out of the fixed bed reactor was separated into gas and liquid to obtain the crude acetonin (liquid). The crude acetonin was analyzed by gas chromatography. The analysis results showed that the selectivity of the acetonin was 100% and the yield of acetonin was 92%.

70 g of the acetonin crude, 3.15 g of water (4.5 wt %), and 8 g of Amberlyst 15 were added into an autoclave reactor, then heated to 75° C. and kept at a pressure of 10 kg/cm² to react for 6 hours to obtain a crude TAA. The crude TAA was analyzed by gas chromatography, which showed the yield of TAA was 83% and the rest were side products. As shown in Example a-5, the acetone obtained by cracking the side products could be reused to prepare the acetonin, and the acetonin could be used to prepare the triacetonamine.

### Example a-6

A fixed bed reactor was set to have an inner diameter of 12.7 mm and a tube length of 760 mm. 20 mL of sulfonic acid resin Amberlyst 15 was filled into the reactor to serve as an acidic catalyst. The fixed bed reactor was maintained at a temperature of 35° C. and maintained at a pressure of 10 kg/cm².

Acetone and liquid ammonia (molar ratio=5.7:1) were introduced into the fixed bed reactor, the LHSV of the acetone was 1.15 h⁻¹, and the LHSV of the liquid ammonia was 0.06 h⁻¹. The crude product flowing out of the fixed bed reactor was separated into gas and liquid to obtain the crude acetonin (liquid). The crude acetonin was analyzed by gas chromatography. The analysis results showed the selectivity of acetonin was 100% and the yield of acetonin was 94%.

### Example a-7

A fixed bed reactor was set to have an inner diameter of 12.7 mm and a tube length of 800 mm. 20 mL of sulfonic acid resin Amberlyst 15 was filled into the reactor to serve as an acidic catalyst. The fixed bed reactor was maintained at a temperature of 75° C. and maintained at a pressure of 10 kg/cm².

Water was added to the crude acetonin in Example a-3 (crude acetonin and water had a weight ratio of 100:4.5), which were introduced into the fixed bed reactor, and the total

LHSV of the acetonin crude and water was 0.6 h⁻¹. The product flowing out of the fixed bed reactor was analyzed by gas chromatography, which showed that the selectivity of TAA was 95% and the rest were side products. As shown in Example a-7, the reactor for reacting the acetonin and water could be not only the autoclave reactor but also the fixed bed reactor.

### Example 7

The TAA crude in Example a-3 was distilled to obtain a light-boiling substance. According to the gas chromatography analysis, the light-boiling substance contained 0.16 parts of acetone, 0.06 parts of MO, 0.51 parts of TMDP, 0.35 parts of ACTN, 0.10 parts of DAAM, 0.41 parts of DAA, and 0.27 parts of TAA (relative to the integrated area (1 part) of an internal standard isopropyl alcohol).

25 g of the light-boiling substance, 20 g of water, and 4 g of the product in Synthesis Example 1 were mixed and then heated to a temperature range of 80° C. to 90° C. to react for 3 hours. The reaction product was analyzed by gas chromatography, which resulted in 1.18 parts by acetone, 0 parts by MO, 1.10 parts by TMDP, 0 parts by ACTN, 0 parts by DAAM, 0 parts by DAA, and 0.32 parts by TAA (relative to the integrated area (1 part) of an internal standard isopropyl alcohol). Accordingly, the product in Synthesis Example 1 could effectively crack the side products of TAA to acetone.

25 g of the light-boiling substance, 20 g of water, and 4 g of sodium hydroxide were mixed and then heated to a temperature of 80° C. to 90° C. to react for 3 hours. The reaction result was analyzed by gas chromatography, resulted in 1.14 parts by acetone, 0 parts by MO, 1.2 parts by TMDP, 0 parts by ACTN, 0 parts by DAAM, 0 parts by DAA, and 0.32 parts by TAA (relative to the integrated area (1 part) of an internal standard isopropyl alcohol). Accordingly, the sodium hydroxide was less effective in cracking the side products of TAA to acetone compared to that of the product in Synthesis Example 1.

### Example 8

The TAA crude in Example a-3 was distilled to obtain a light-boiling substance. According to the gas chromatography analysis, the light-boiling substance contained 0.34 parts of acetone, 0.05 parts of MO, 0.63 parts of TMDP, 1.02 parts of ACTN, 0.32 parts of DAAM, 0.69 parts of DAA, and 0.48 parts of TAA (relative to the integrated area (1 part) of an internal standard isopropyl alcohol).

25 g of the light-boiling substance, 20 g of water, and 4 g of the product in Synthesis Example 2 were mixed and then heated to a temperature range of 80° C. to 90° C. to react for 3 hours. The reaction product was analyzed by gas chromatography, which resulted in 2.12 parts by acetone, 0 parts by MO, 1.84 parts by TMDP, 0 parts by ACTN, 0 parts by DAAM, 0 parts by DAA, and 0.52 parts by TAA (relative to the integrated area (1 part) of an internal standard isopropyl alcohol). Accordingly, the product in Synthesis Example 2 could effectively crack the side products of TAA to acetone.

25 g of the light-boiling substance, 20 g of water, and 4 g of sodium hydroxide were mixed and then heated to a temperature range of 80° C. to 90° C. to react for 3 hours. The reaction result was analyzed by gas chromatography, which resulted in 1.62 parts by acetone, 0 parts by MO, 2.06 parts by TMDP, 0 parts by ACTN, 0 parts by DAAM, 0 parts by DAA, and 0.61 parts by TAA (relative to the integrated area (1 part) of an internal standard isopropyl alcohol). Accordingly, the sodium hydroxide was less effective in cracking the side products of TAA to acetone compared to that of the product in Synthesis Example 2.

It will be apparent to those skilled in the art that various modifications and variations can be made to the disclosed methods and materials. It is intended that the specification and examples be considered as exemplary only, with the true scope of the disclosure being indicated by the following claims and their equivalents.

What is claimed is:

1. A method of manufacturing triacetonamine, comprising:
(a) introducing an acetone and an ammonia into a first reactor in the presence of a first acidic catalyst to form an acetonin;
(b) introducing the acetonin and water into a second reactor in the presence of a second acidic catalyst to form a triacetonamine and side products, wherein the side products are diacetone alcohol, diacetone amine, mesityl oxide, 2,2,4,6-tetramethyl-2,3-dihydropyridine, or a combination thereof;
(c) separating the triacetonamine and the side products by distillation under reduced pressure;
(d) introducing the side products and water into a third reactor to heat and crack the side products in the presence of an amphiphilic catalyst to form acetone; and
(e) introducing the acetone obtained from step (d) into the first reactor.

2. The method as claimed in claim 1, wherein the amphiphilic catalyst has a chemical structure of 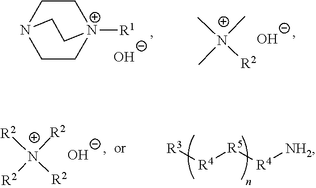
wherein R¹ is C₄₋₁₀ alkyl group, each of R² is independently C₄₋₁₆ alkyl group, R³ is —NH₂ or —OH, R⁴ is C₂₋₆ alkylene group, R⁵ is —NH— or —O—, and n=0 to 4.

3. The method as claimed in claim 1, wherein the amphiphilic catalyst and the side products have a molar ratio of 1:5 to 1:40.

4. The method as claimed in claim 1, wherein a reaction temperature of the first reactor ranges between 20° C. to 40° C.

5. The method as claimed in claim 1, wherein a reaction temperature of the second reactor ranges between 60° C. to 90° C.

6. The method as claimed in claim 1, wherein the acetone and the ammonia of step (a) have a molar ratio of 1:1 to 20:1.

7. The method as claimed in claim 1, wherein a reaction temperature of the third reactor ranges between 70°° C. to 90° C.

8. A device for manufacturing triacetonamine, comprising:
a first reactor containing a first acidic catalyst and receiving an acetone and an ammonia to form an acetonin;
a second reactor containing a second acidic catalyst and connected to the first reactor and a water source for receiving the acetonin and water, respectively, to form a triacetonamine and side products, wherein the side products are diacetone alcohol, diacetone amine, mesityl oxide, 2,2,4,6-tetramethyl-2,3-dihydropyridine, or a combination thereof;
a distiller connected to the second reactor for receiving the triacetonamine and the side products and separating the triacetonamine and the side products by distillation under reduced pressure; and
a third reactor containing an amphiphilic catalyst and connected to the distiller and the water source for receiving the side products and water, respectively, to heat and crack the side products to form an acetone,
wherein the third reactor is connected to the first reactor for introducing the acetone in the third reactor into the first reactor.

9. The device as claimed in claim 8, wherein the amphiphilic catalyst has a chemical structure of 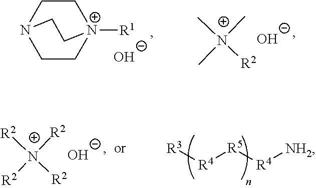
wherein R¹ is C₄₋₁₀ alkyl group, each of R² is independently C₄₋₁₆ alkyl group, R³ is —NH₂ or —OH, R⁴ is C₂₋₆ alkylene group, R⁵ is —NH— or —O—, and n=0 to 4.
